(19) United States
(12) Patent Application Publication
(10) Pub. No.: US 2013/0218008 A1
(43) Pub. Date: Aug. 22, 2013

(54) MEASURING DEVICE, MEASURING METHOD, AND TOMOGRAPHIC APPARATUS

(71) Applicant: CANON KABUSHIKI KAISHA, Tokyo (JP)

(72) Inventor: Takeaki Itsuji, Hiratsuka-shi (JP)

(73) Assignee: CANON KABUSHIKI KAISHA, Tokyo (JP)

(21) Appl. No.: 13/769,170

(22) Filed: Feb. 15, 2013

(30) Foreign Application Priority Data
Feb. 20, 2012 (JP) 2012-034396

(51) Int. Cl. A61B 5/00

(52) U.S. Cl. CPC A61B 5/0073; A61B 5/7257
USPC 600/425; 600/476

(57) ABSTRACT

A measuring device for measuring a physical property of an object which is irradiated with an electromagnetic wave pulse. The measuring device includes a waveform obtaining unit which obtains a time waveform from a signal relating to the electromagnetic wave pulse reflected from a first reflection portion and a second reflection portion of the object. The waveform obtaining unit obtains a first obtained waveform at a first collection point where a parallel region of the electromagnetic wave pulse is adjusted by a position adjusting unit so as to be in only one of the first reflection portion and the second reflection portion of the object, and obtains a second obtained waveform at a second collection point different from the first collection point. A waveform forming unit forms a measured waveform based on the first obtained waveform and the second obtained waveform.

### BACKGROUND OF THE INVENTION

1. Field of the Invention

The present invention relates to a measuring device, measuring method, and a tomographic apparatus for measuring a physical property using an electromagnetic wave.

2. Description of the Related Art

A characteristic absorption spectrum based on the structure and state of a substance, including a biomolecule, in a frequency band of a predetermined electromagnetic wave, can be typified by a terahertz wave. Terahertz waves are electromagnetic waves in a frequency band in a range from millimeter waves to terahertz waves, in particular, a frequency band from 0.03 THz to 30 THz.

An inspection technique that makes use of such a characteristic absorption spectrum for analyzing or identifying a substance nondestructively using an electromagnetic wave of a predetermined frequency band of the terahertz domain has been developed. This inspection technique is expected to be applied to an imaging technique that substitutes or augments an X-ray imaging technique, and to a high-speed communication technique. In addition, attention has been given to application of terahertz waves to a tomographic apparatus that visualizes the inside of an object using a reflected terahertz wave pulse from a refractive index interface inside the object. The tomographic apparatus using a terahertz wave pulse is expected to visualize the internal structure at a depth of from several hundred micrometers to several tens of millimeters by making use of the transmission of a terahertz wave.

In time-domain spectroscopy, which is often used in such a tomographic apparatus, an electromagnetic wave pulse is subjected to sampling measurement using ultra short pulse light (hereinafter also referred to as excitation light) having a pulse width in the order of femtoseconds (1×10⁻¹⁵ sec.). Sampling measurement on an electromagnetic wave pulse is made by sampling while adjusting an optical path length difference of excitation light that reaches a generation unit that generates the electromagnetic wave pulse and a detection unit that detects the electromagnetic wave pulse. A known method of adjusting an optical path length difference is to adjust it on the basis of the amount of folding of excitation light using a stage that includes a folding optical system, the stage being inserted into an optical path (propagation route) of the excitation light. Another known method is the one using, in the generation unit or detection unit, a photoconductive element in which an antenna electrode pattern including a minute gap in a semiconductor thin film.

Japanese Patent application Laid-Open No. 2004-28618 discloses a biosensor measuring device that calculates the thickness of a coating film from a change in interval between a plurality of electromagnetic wave pulses reflected from an interface in an object using such a time-domain spectroscopic principle. This device obtains a frequency spectrum for each extracted reflected pulse and calculates an absorption spectrum from a ratio among frequency spectrums by extracting a reflected pulse in each electromagnetic wave pulse on a temporal axis and then performing Fourier transform.

In the case where a reflected-pulse signal from a certain interface is extracted from a time waveform of a detected reflected pulse and a physical property is obtained, it is difficult to have sufficient measurement time length, resolution is coarse, and the accuracy of measurement of the physical property of an object may decrease. This is because the resolution for an object is dependent on the time length of a detected electromagnetic wave pulse.

### SUMMARY OF THE INVENTION

Accordingly, there is a need to increase accuracy in the measurement of a physical property of an object even when electromagnetic waves are reflected from a plurality of interfaces in an object. Embodiments in the present application are directed to addressing this and other needs.

A measuring device according to embodiments of the present invention has a configuration described below.

That is, embodiments of the present invention are directed to a measuring device for measuring a physical property of an object which is irradiated by an electromagnetic wave pulse, the object including a first reflection portion and a second reflection portion. The measuring device includes a detecting unit configured to detect the electromagnetic wave pulse, an optical delaying unit configured to delay an optical path length of excitation light reaching the detecting unit or the electromagnetic wave pulse, a collecting unit configured to collect the electromagnetic wave pulses to a collection point, a position adjusting unit configured to adjust a positional relationship between the object and the collection point such that a depth of focus of the electromagnetic wave pulse is in at least one of the first reflection portion and the second reflection portion of the object, a waveform obtaining unit configured to change the optical path length in the optical delaying unit, obtain a time waveform from a signal relating to the electromagnetic wave detected by the detecting unit, obtain a first obtained waveform at a first collection point where the depth of focus of the electromagnetic wave pulse is adjusted by the position adjusting unit so as to be in only one of the first reflection portion and the second reflection portion, and obtain a second obtained waveform at a second collection point different from the first collection point, and a waveform forming unit configured to form a measured waveform based on the first obtained waveform and the second obtained waveform.

A measuring method according to embodiments of the present invention is described below.

That is, the measuring method is used in a measuring device for measuring a physical property of an object, the object including a first reflection portion and a second reflection portion. The measuring device includes a detecting unit configured to detect an electromagnetic wave pulse, an optical delaying unit configured to delay an optical path length of excitation light reaching the detecting unit, a collecting unit configured to collect the electromagnetic wave pulses to a collection point, a position adjusting unit configured to adjust a positional relationship between the object and the collection point such that a depth of focus of the electromagnetic wave pulse is in at least one of the first reflection portion and the second reflection portion of the object, a waveform obtaining unit configured to change the optical path length in the optical delaying unit and obtain a time waveform from a signal relating to the electromagnetic wave detected by the detecting unit. The measuring method includes obtaining a first obtained waveform at a first collection point where the depth of focus of the electromagnetic wave pulse is adjusted by the position adjusting unit so as to be in only one of the first reflection portion and the second reflection portion and obtaining a second obtained waveform at a second collection point different from the first collection point, and adjusting positions of first reflected pulses reflected from the first reflection portion in the first and second obtained waveforms on a time axis to respective reference positions to form first and second adjusted waveforms and forming a measured waveform by summing the first and second adjusted waveforms.

A tomographic apparatus according to embodiments of the present invention has a configuration described below.

That is, the tomographic apparatus for obtaining a tomographic image of an object, the object including a first reflection portion and a second reflection portion, the tomographic apparatus includes a detecting unit configured to detect an electromagnetic wave pulse, an optical delaying unit configured to delay excitation light reaching the detecting unit, a collecting unit configured to collect the electromagnetic wave pulses to a collection point, a position adjusting unit configured to perform movement in parallel with an optical axis of the electromagnetic wave pulse in the collection point with respect to the object such that a depth of focus of the electromagnetic wave pulse is in at least one of the first reflection portion and the second reflection portion of the object, a waveform obtaining unit configured to make the optical path length in the optical delaying unit variable, obtain a time waveform from information on the electromagnetic wave detected by the detecting unit, obtain a first obtained waveform at a first collection point where the depth of focus of the electromagnetic wave pulse is adjusted by the position adjusting unit so as to be in only one of the first reflection portion and the second reflection portion, and obtain a second obtained waveform at a second collection point different from the first collection point, a waveform forming unit configured to form a measured waveform based on the first obtained waveform and the second obtained waveform, a stage that holds the object and that relatively moves the object and the position of the electromagnetic wave pulse; and an image constructing unit configured to construct a tomographic image of the object based on the position of the stage and the measured waveform formed by the waveform forming unit.

Further features of the present invention will become apparent from the following description of exemplary embodiments with reference to the attached drawings.

### BRIEF DESCRIPTION OF THE DRAWINGS

FIG. 1 illustrates a physical property measuring device according to a first embodiment.

FIG. 2 illustrates a physical property measuring device according to a second embodiment.

FIG. 3 is a flowchart of a measuring operation of the physical property measuring device according to the first embodiment.

FIG. 4A is an illustration for describing a positional relationship between a beam shape of a terahertz wave pulse and an object, and FIG. 4B is an illustration for describing a time waveform of a terahertz wave pulse obtained from an object.

FIG. 5A is an illustration for describing a relationship between a collection point and a time interval of an electromagnetic wave pulse, and FIG. 5B illustrates a result of an experiment on a collection point and an electromagnetic wave pulse.

FIG. 6A is an illustration for describing a first obtained waveform and a second obtained waveform obtained by a waveform obtaining unit according to the first embodiment, FIG. 6B is an illustration for describing an adjusted waveform adjusted in a waveform adjusting unit, and FIG. 6C is an illustration for describing an extracted measured waveform.

FIG. 7 illustrates a physical property measuring device according to a third embodiment.

FIG. 8 illustrates a physical property measuring device according to a fourth embodiment.

FIG. 9 is a flowchart of a measuring operation in the physical property measuring device according to the third embodiment.

FIG. 10A is a schematic diagram of a skin as an object, and FIG. 10B is a schematic diagram of a skin that includes a cancer tissue as an object.

FIG. 11A is a schematic diagram of a skin that includes a cancer tissue, and FIG. 11B illustrates a tomographic image after measurement of the skin including the cancer tissue.

FIG. 12 illustrates a tomographic image of the skin including the cancer tissue after correction.

### DESCRIPTION OF THE EMBODIMENTS

### First Embodiment

A physical property measuring device according to a first embodiment is described below with reference to the drawings.

### General Configuration of Physical Property Measuring Device

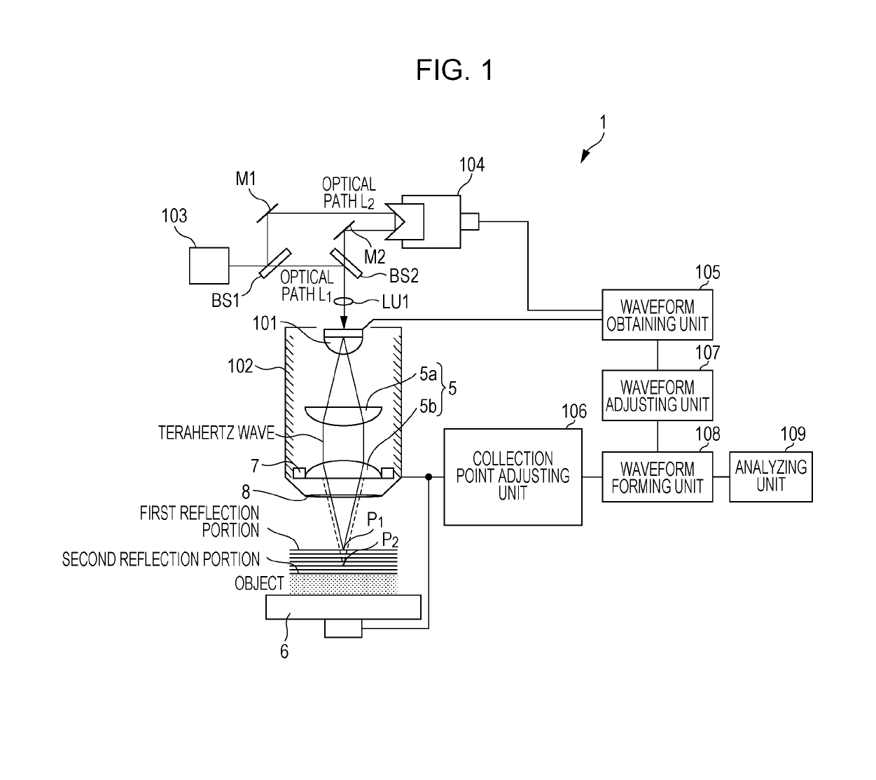

FIG. 1 illustrates a physical property measuring device 1 according to the present embodiment.

The physical property measuring device 1 includes a light source 103, a generating and detecting unit 101 that generates and detects an electromagnetic wave pulse, and a shaping unit 102 that collects and shapes an electromagnetic wave pulse and measures a physical property of an object. The light source 103 emits excitation light (laser light) for use in generating and detecting an electromagnetic wave pulse by the generating and detecting unit 101.

The physical property measuring device 1 further includes a waveform obtaining unit 105, a collection point adjusting unit 106, a waveform adjusting unit 107, a waveform forming unit 108, and an analyzing unit 109.

The waveform obtaining unit 105 obtains a time waveform of a reflected pulse reflected from an object on the basis of a result of detection performed by the detecting unit. In certain embodiments, the waveform obtaining unit 105 may be implemented by hardware (e.g., digital oscilloscope), or software implemented in hardware (e.g., an algorithm executed by a microprocessor or computer). The collection point adjusting unit 106 moves and adjusts a position where electromagnetic wave pulses are collected with respect to the object. The waveform adjusting unit 107 moves and adjusts a position of the time waveform on a time axis obtained by the waveform obtaining unit 105. The waveform forming unit 108 forms a time waveform (extracted waveform) from a desired interface by referring to the time waveform output from the waveform adjusting unit 107 in response to a change in the collection point and adding the adjusted waveform. The analyzing unit 109 analyzes the object on the basis of the time waveform obtained in the waveform forming unit 108. The components are further described below.

### Light Source

The light source 103 outputs excitation light (laser light) toward the generating and detecting unit 101. The laser light output from the light source 103 has a pulse width of several tens of femtoseconds. A photoconductive element that forms the generating and detecting unit 101 produces a terahertz wave by excitation of a carrier in a semiconductor thin film by radiation with excitation light.

As illustrated in FIG. 1, excitation light output from the light source 103 is split, by a beam splitter BS1, into a first optical path L₁ and a second optical path L₂. Excitation light passing along the optical path L₁ is directed toward the generating and detecting unit 101 via a beam combiner BS2 and a lens unit LU1. Excitation light traveling along optical path L₁ is used as excitation light for generating a pulsed terahertz wave (terahertz wave pulse). Excitation light passing along the optical path L₂ is directed toward the generating and detecting unit 101 by a series of mirrors M1 and M2 through an optical delaying unit 104 (delay unit), the beam combiner BS2 and the lens unit LU1. The excitation light traveling along optical path L₂ is used as excitation light for detection of a terahertz wave pulse. The wavelength of the excitation light output from the light source 103 is determined by an absorption wavelength of the semiconductor film of a photoconductive element used in the generating and detecting unit 101. As used herein, a “photoconductive element” generally refers to certain semiconductor materials or compounds thereof, which when irradiated with an ultra short laser pulse (100 femtoseconds or shorter), are capable of abruptly changing from insulator to conductor to thereby generate short-lived charge carriers (electron-hole pairs).

Two laser sources for outputting excitation light traveling along optical path L₁ and excitation light traveling along optical path L₂ may be used as the light source 103. The wavelength, pulse width and a pulse repetition frequency (pulse rate) of light output from the light source 103 (laser) can be selected depending on device specifications necessary for specific applications.

### Optical Delaying Unit

The optical delaying unit 104 adjusts the optical path length of excitation light and adjusts the optical path length difference between the optical path L₁ and optical path L₂ reaching the generating and detecting unit 101. That is, in the present embodiment, the optical path length of excitation light is increased so as to delay arrival at the generating and detecting unit.

It is difficult to detect a terahertz wave pulse in real time. Thus in terahertz time-domain spectroscopy, the optical path length difference between the excitation light propagating through optical path L₁ and excitation light propagating through optical path L₂ directed to the generating and detecting unit 101 is changed by every predetermined amount of the optical path length, and a terahertz wave pulse is subjected to sampling measurement. This adjustment can use a technique of directly adjusting a physical optical path length (distance traveled) of excitation light or a technique of adjusting an effective optical path length.

The technique of directly adjusting the optical path length is to adjust the path or distance traveled by the excitation light by moving a folding optical system for folding excitation light in a direction along a folding optical path. The technique of adjusting the effective optical path length is to adjust it by changing a time constant in the length of an optical path along which excitation light propagates (for example, the effective optical path length can be adjusted by changing the refractive index in the optical path). Both techniques can be interchangeably used in the present embodiment.

### Generating and Detecting Unit

The generating and detecting unit 101 includes a photoconductive element that serves as both a generating unit and a detecting unit for an electromagnetic wave (also referred to as a terahertz wave) containing a part of a frequency band of, in particular, from 30 GHz to 30 THz. The generating and detecting unit 101 produces a terahertz wave pulse, which is an electromagnetic wave pulse, by being radiated with excitation light, and detects a terahertz wave pulse (reflected pulse) that is an electromagnetic wave pulse reflected from an object.

Here, detecting an electric field strength of a terahertz wave using a current (instantaneous current) output from the photoconductive element is used as the method of detecting a terahertz wave pulse in the generating and detecting unit 101. A photoconductive element in which an antenna pattern is formed on a semiconductor film using a metal electrode can be used as the element for detecting the current. A method of detecting an electric field of an antenna pattern employing the electro-optical effect or a method of detecting a magnetic field of an antenna pattern employing the magneto-optical effect is also applicable.

For a principle of generating a terahertz wave pulse, a terahertz wave is produced by radiation of a surface of a semiconductor or a nonlinear crystal with excitation light. When a photoconductive element is used, radiating the photoconductive element being in the state where an electric field is applied to an electrode of the photoconductive element with excitation light generates a terahertz wave. When the electro-optical effect of a nonlinear optical crystal is used, polarization occurring in the crystal resulting from radiation with excitation light generates a terahertz wave. When an instantaneous current is used, a PIN diode structure may be employed. A technique that utilizes an interband transition of a charge carrier (electron-hole pair) may also be employed.

The generating unit and detecting unit for a terahertz wave may be provided as separate units. Depending on the wavelength of excitation light (e.g. depending on the spectrum portion used for terahertz generation, and on the spectrum portion used for terahertz detection), a wavelength converting element may be disposed in the optical path L₁ or optical path L₂.

### Beam Shaping Unit

The shaping unit 102 adjusts a beam shape of a terahertz wave pulse and collects the terahertz wave pulses. That is, it can adjust a beam shape and move a collection point of a terahertz wave pulse on the optical axis.

The shaping unit 102 includes, for example, two lenses 5a and 5b constituting a collecting unit 5 configured to adjust a beam shape of a terahertz wave pulse and collect the terahertz wave pulses. A housing 8 having an exit window houses the two lenses 5a and 5b and the generating and detecting unit 101. Any other configurations that can collect terahertz wave pulses to an object may also be used. For example, the housing with the window may not be used. The shaping unit 102 collects (focuses) the terahertz wave pulses to a collection point using the two lenses 5a and 5b. The collecting unit 5 may be composed of the two lenses (as shown), a single lens, or three or more lenses. The shaping unit 102 includes an actuator 7. The actuator 7 is a moving unit that moves the window-side lens in a direction parallel to the direction in which terahertz wave pulses propagate (to the optical axis direction). An object, mounted in a movable stage 6, is disposed along the optical axis direction.

Adjusting the position of the window-side lens 5b can adjust the position where terahertz waves are collected. When the shaping unit 102 houses the generating and detecting unit 101, a mechanism in which the shaping unit 102 itself moves in the direction of propagation of terahertz wave pulses may be included. The collecting unit 5 may include a mirror, instead of a lens.

Beam shapes of terahertz wave pulses collected by the collecting unit 5 can be broadly divided into a region where collection of terahertz wave pulses is in progress (hereinafter referred to as collection-in-progress region A) and a region where terahertz wave pulses corresponding to the depth of focus are considered to propagate in parallel with each other (hereinafter referred to as parallel region B). The details of the regions are described below.

### Waveform Obtaining Unit

The waveform obtaining unit 105 changes an optical path length in the optical delaying unit and obtains a time waveform of a terahertz wave pulse on the basis of a signal relating to the terahertz wave pulse detected in the generating and detecting unit 101. Because a terahertz wave pulse typically has a pulse waveform with a pulse width on the order of picoseconds or less, it is difficult to obtain the terahertz wave pulse in real time. Thus optical sampling that can measure a pulse width shorter than the pulse width of the terahertz wave pulse is performed.

When a photoconductive element is used in the generating and detecting unit 101, as in the present embodiment, excitation light emitted from the light source 103 is used as pulse light in the optical sampling measurement. The excitation light in the present embodiment is pulse light having a pulse width of femtoseconds. The sampling measurement for a terahertz wave pulse is made by changing the length of the optical path L₂ in the optical delaying unit 104 and adjusting the optical path length difference between a terahertz wave pulse reaching the generating and detecting unit 101 via optical path L₁ and that of the optical path L₂.

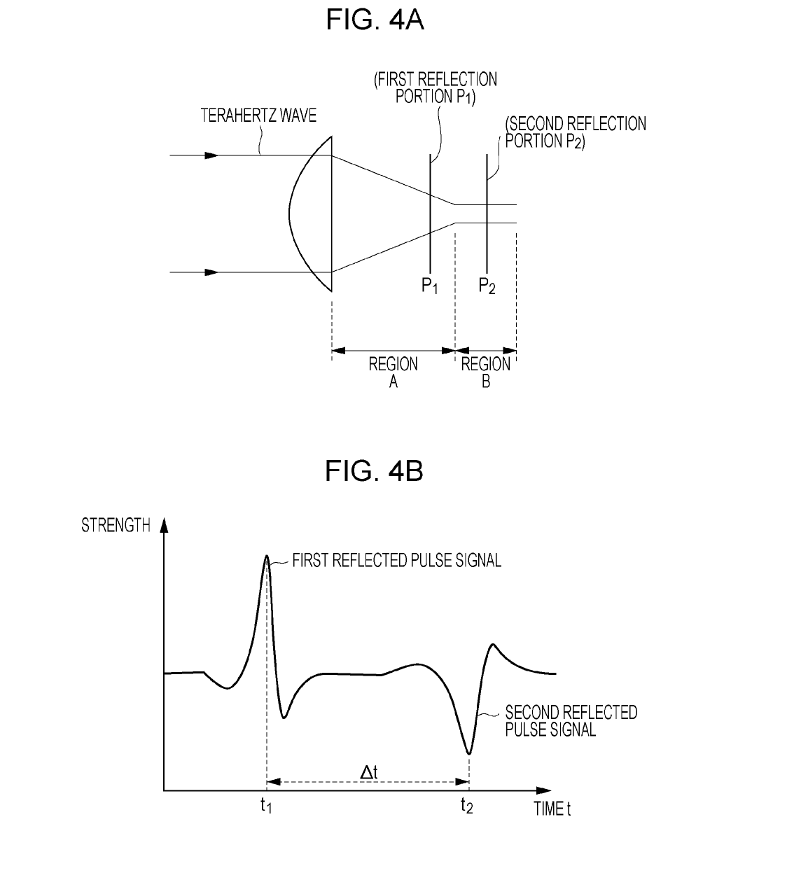

The waveform obtaining unit 105 establishes a time waveform of a terahertz wave pulse using the amount of adjustment of the optical path length of a terahertz wave pulse reaching the generating and detecting unit 101 in the optical delaying unit 104 and a detection signal of a reflected terahertz pulse corresponding to that amount of adjustment obtained in the generating and detecting unit 101. When an object includes a plurality of interfaces, for example, it includes a first reflection portion and a second reflection portion, a time waveform of a terahertz wave pulse established in the waveform obtaining unit contains a first reflection signal from the first reflection portion and a second reflection signal from the second reflection portion, as illustrated in FIG. 4B.

### Collection Point Adjusting Unit

The collection point adjusting unit 106 is a position adjusting unit that moves a focal point of a terahertz wave pulse (collection point) along a direction substantially along the optical axis of the terahertz wave pulse and that matches the collection point with a desired position. For example, the collection point adjusting unit 106 can adjust the collection point of a produced terahertz wave pulse by moving it from a first collection point P₁ to a second collection point P₂.

Each of the first collection point P₁ and second collection point P₂ is represented as a collection point indicated by one point, but it can be a region where light is considered to be focused, that is, a parallel region that corresponds to the depth of focus. The collection point of a terahertz wave pulse is adjusted by movement of a lens in the collecting unit 5 while the position of an object is fixed. The collection point in the object may be adjusted by movement of the object in a direction substantially along the optical axis of a terahertz wave in the collection point by the actuator 7.

### Analyzing Unit

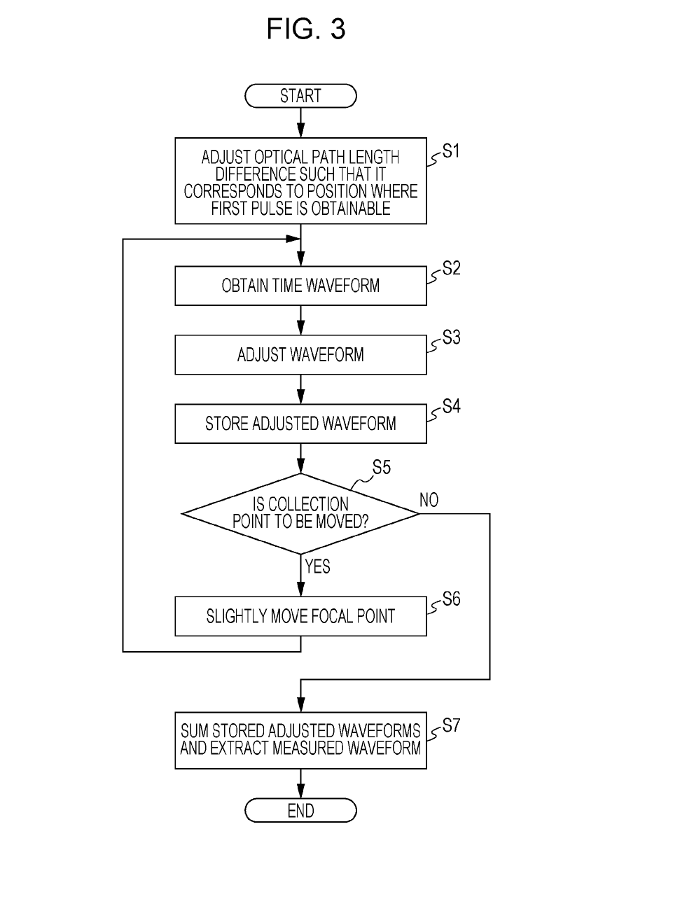

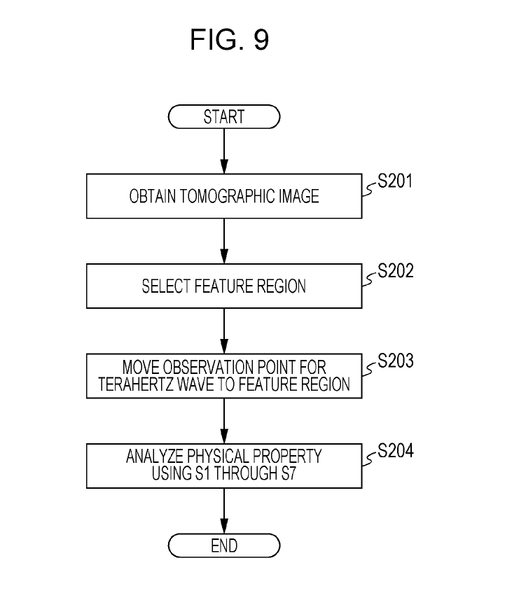

The analyzing unit 109 includes a storage unit and a comparing unit (both not shown). The storage unit (e.g., a memory) is configured to store information on a previously measured physical property. The comparing unit is configured to compare information on a measured physical property with the stored physical property information. The analyzing unit 109 analyzes the physical property of a reflection portion of the object of interest. For example, a refractive index distribution and an absorption coefficient of the object are obtained by monitoring a change in a reflected terahertz wave pulse from reference information. Alternatively, the physical property of the object can also be analyzed by comparison of a change in a frequency spectrum or time waveform with a previously prepared database of the object. As used herein, the analyzing unit 109 including the storage unit and the comparing unit (both not shown) may be implemented by hardware, software, or a combination of both. More specifically, the analyzing unit 109 including the storage unit and comparing unit (both not shown) may be implemented by a general purpose computer including at least one microprocessor (CPU) and a memory device (hard disk drive or removable RAM), which may be programmed with specific algorithms (program code) to collectively execute the processes illustrated by flow diagrams of FIGS. 3 and 9, among others.

### Method of Measuring Object for Use in Physical Property Measuring Device

A relationship between a beam shape of terahertz wave pulses collected by the collecting unit and a reflection portion of an object is described with reference to FIGS. 4A and 4B. FIG. 4A is an illustration of a terahertz wave being collected (focused) for describing a positional relationship between a beam shape of a terahertz wave pulse and an object. FIG. 4B is a spectral graph in Cartesian coordinates illustrating a time waveform of a terahertz wave pulse obtained from an object.

As illustrated in FIGS. 4A and 4B, a terahertz wave pulse is considered to include broadly divided two regions. Of the two regions, the region where terahertz wave pulses are collected in progress is referred to as the collection-in-progress region A (hereinafter region A), and the region where terahertz wave pulses propagate in parallel with each other is referred to as the parallel region B (hereinafter region B). The parallel region B corresponds to the depth of focus in terms of wave optics.

When the gap between the first reflection portion and the second reflection portion (special gap) is t and a mean refractive index between the first reflection portion and the second reflection portion is n, the optical path length of a terahertz wave pulse that propagates from the first reflection portion to the second reflection portion is approximated to t×n. When the collection point is moved from the first reflection portion to the second reflection portion by the collection point adjusting unit 106, the amount of movement of the collection point is approximated to t/n. For simplification of description, the amount of movement of the parallel region and the amount of movement of the collection point are assumed to be equal in the present embodiment.

When the reflection portion of the object moves in the parallel region (region B), because an electromagnetic wave pulse is considered to be focused, the beam shape of a reflected pulse wave reaching the generating and detecting unit 101 remains substantially unchanged. Thus the optical movement distance of the electromagnetic wave pulse in the parallel region together with movement of the reflection portion is approximately proportional to a relative movement distance with respect to the reflection portion.

When the reflection portion in the object moves in the collection-in-progress region A (region A), because it is out of focus for an electromagnetic wave pulse, for the beam shape of a terahertz wave reaching the generating and detecting unit 101, an angle component resulting from enlargement and reduction of the beam diameter is added to the distance of movement of the reflection portion. The optical movement distance of the reflection portion at this time is longer than the optical movement distance in the parallel region. The optical movement distance can be converted into a propagation time of a terahertz wave pulse. For the time waveform obtained in the waveform obtaining unit 105, reflected pulses from the first reflection portion and second reflection portion of the object are detected. Depending on in which region for an electromagnetic wave pulse each of the reflection portions is present, the time interval Δt changes. The time interval Δt is the time difference between a reflected pulse from the first reflection portion and that from the second reflection portion in FIG. 4B. A measured waveform in the present embodiment is one in which a time waveform of a predetermined reflected pulse is extracted on the basis of the first and second obtained waveforms obtained by changing the time interval Δt.

In the present embodiment, a time waveform of an electromagnetic wave pulse from the first collection point P₁ is referred to as a first obtained waveform, and a time waveform of an electromagnetic wave pulse from the second collection point P₂ is referred to as a second obtained waveform. For the sake of convenience, time waveforms of electromagnetic wave pulses reflected from two collection points are used in the description for the present embodiment, and the number of these time waveforms is equal to the number of the collection points. Specifically, the first obtained waveform and second obtained waveform indicate that collection points at the time of measurement of time waveforms are different.

The time interval Δt between a first reflected pulse and a second reflected pulse included in the first obtained waveform and the second obtained waveform varies depending on whether the reflection portion of the object corresponds to the collection-in-progress region or parallel region. In other words, when the collection point of terahertz wave pulses is changed by the collection point adjusting unit 106, the time interval Δt changes. Changes in the time interval Δt can be in three states described below.

A first state is the state in which the first reflection portion and second reflection portion of the object are within the collection-in-progress region (region A) and the collection point for the object changes such that the first and second reflection portions are within this region (hereinafter also referred to as collection-in-progress region A). In this state, the change in the time interval Δt in FIG. 4B is small.

When the collection point is changed from the first collection point P₁ to the second collection point P₂, as illustrated in FIG. 1, the time interval Δt changes by the amount reflecting the difference between the amounts of changes in the optical path length at the collection points. That is, the optical path length of a terahertz wave pulse slightly changes in accordance with enlargement or reduction in the beam diameter of the terahertz wave pulse reaching the generating and detecting unit 101.

A second state is the state in which the first reflection portion and second reflection portion of the object are within the parallel region (region B) and the collection point for the object changes such that the first and second reflection portions are within this region (hereinafter also referred to as parallel region B). In this state, the change in the time interval Δt is extremely small (and may be considered negligible) because the optical movement distance of each of the first and second reflection portions is approximately proportional to the physical movement distance.

A third state is the state in which one of the first reflection portion and second reflection portion of the object is within the collection-in-progress region A and the other is within the parallel region B, that is, the collection point for the object changes such that only one of the first and second reflection portions is within the parallel region.

The state in which one of the first and second reflection portions is within the collection-in-progress region A and the other is within the parallel region B is hereinafter referred to as a mixed region A+B. In addition to the amount of physical movement of the reflection portion, the amount of change in the optical path length resulting from enlargement or reduction in the beam diameter of a terahertz wave pulse reaching the generating and detecting unit 101 is reflected in the position of a reflected pulse from the reflection portion in the collection-in-progress region A on the time axis, as described above. In contrast, the position of a reflected pulse from the reflection portion in the parallel region B on the time axis reflects the amount of physical movement of the reflection portion. As a result, the amount of change in the optical path length resulting from a change in the beam shape directly acts on the time interval Δt in FIG. 4B. In the present embodiment, the object in the mixed region A+B is a target, and the reflected pulse from the reflection portion of interest is formed using the change in the time interval Δt.

### Adjustment of Collection Point of Terahertz Wave Pulse

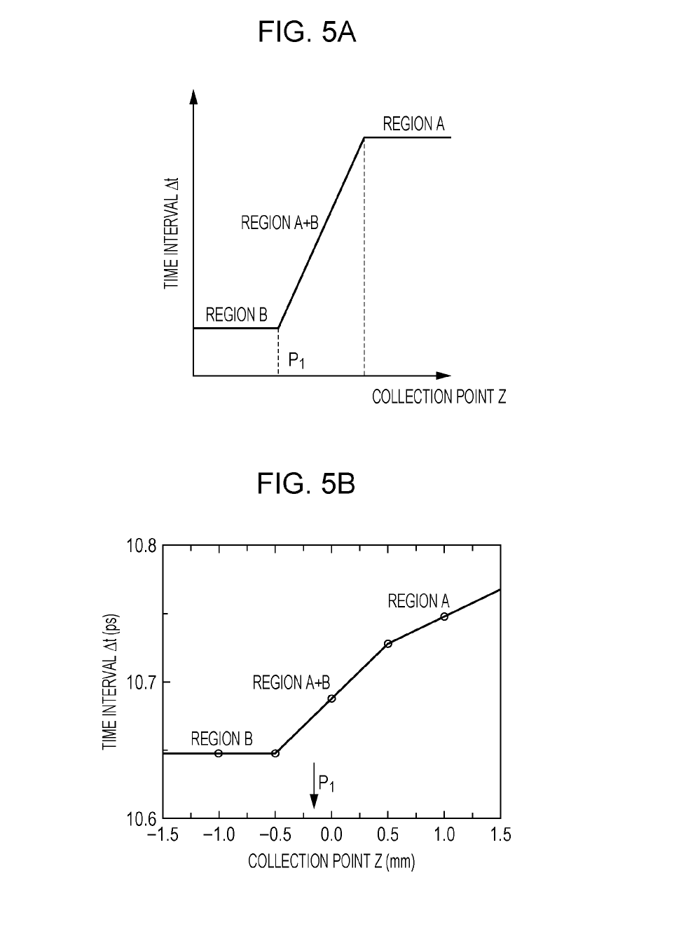

A collection point Z of a terahertz wave pulse and the time interval Δt between reflected pulses are described below. FIG. 5A is a concept diagram that illustrates a relationship between the collection point Z of a terahertz wave pulse and the time interval Δt between reflected pulses. When both the first reflection portion and the second reflection portion are positioned in the collection-in-progress region A or the parallel region B, the time interval Δt is not substantially dependent on the collection point Z, and the time interval Δt is substantially constant even when the collection point Z is changed.

In contrast, when the first reflection portion and the second reflection portion are in the mixed region A+B, the time interval Δt changes with a change in the position of the collection point Z. That is, when the value of the collection point Z increases (collection point Z moves), the time interval Δt also lengthens; when the value of the collection point Z reduces, the time interval Δt also shortens. Here, the direction in which the collection point Z increases is the direction in which the focal length lengthens and the direction from collection point P₁ to collection point P₂ in FIG. 1.

FIG. 5B illustrates a result of experiment of a change in the time interval Δt between reflected pulses with respect to a change in the collection point Z. A change in the time interval Δt between reflected pulses when an object that includes polyethylene, quartz, and an air layer disposed therebetween is plotted. The thickness corresponding to the distance between the top surface and bottom surface of the object is approximately 1.1 mm.

The result of experiment illustrated in FIG. 5B reveals that a change in the time interval Δt between the first reflected pulse and the second reflected pulse with respect to a change in the collection point Z has a tendency similar to that illustrated in FIG. 5A. That is, when the object is positioned in the mixed region A+B, a change in the time interval Δt is large with respect to a change in the collection point Z. In contrast, when both are positioned in the collection-in-progress region A or parallel region B, a change in the time interval Δt is smaller than that occurring when both are in the mixed region A+B.

According to the result of experiment illustrated in FIG. 5B, when the collection point Z changes in the collection-in-progress region A, the time interval Δt also changes. This reflects a difference of the amount of change in optical path length resulting from a change in the beam shape of a terahertz wave pulse reaching the generating and detecting unit 101.

In the present embodiment, a time waveform of a reflected pulse from the reflection portion of interest is formed using a phenomenon in which the time interval between the first reflected pulse and the second reflected pulse changes with respect to the collection point. That is, the collection point of terahertz wave pulses is adjusted such that the first and second reflection portions of the object are present in the mixed region A+B or both of the first and second reflection portions are present in the collection-in-progress region. Here, the collection point may preferably be adjusted such that the first and second reflection portions are present in the mixed region. The details are described below.

The parallel region B, mixed region A+B, and collection-in-progress region A are determined in a way described below. The parallel region B corresponds to the depth of focus and is the region where terahertz waves are considered to propagate in parallel with each other. In the parallel region B, because the time interval Δt between the first reflected pulse and the second reflected pulse does not change when the collection point of terahertz wave pulses changes, the collection point where the time interval Δt starts changing is the boundary between the parallel region B and the mixed region A+B. The mixed region A+B and the collection-in-progress region A have different amounts of change in the time interval Δt with respect to a change in the collection point. The position where the amount of change varies is the boundary between the mixed region A+B and the collection-in-progress region A.

It has been known that the depth of focus, which is the limit range where the object is in focus on the optical axis, is approximated to nλ/2(NA)², where λ is the wavelength of an electromagnetic wave, n is the refractive index, and NA is the numerical aperture of the optical system. The depth of focus of a terahertz wave pulse is typically in the range from 0.1 to 10 mm and is approximately 1 mm in the configuration in the present embodiment.

In the present embodiment, a region upstream of the parallel region B in the propagating direction of an electromagnetic wave pulse is illustrated and described as the collection-in-progress region A. Even when the first reflection portion or the second reflection portion of the object is contained in a region downstream of the parallel region B in the propagating direction of an electromagnetic wave, this situation can be considered to be the mixed region A+B.

The boundary between the parallel region B and the collection-in-progress region A may be established or determined in advance, and it may be stored in memory. Indeed, the boundaries of the parallel region B, mixed region A+B, and collection-in-progress region A for each object may be established or determined before measurement.

### Adjustment of Obtained Reflected Pulse

The waveform adjusting unit 107 adjusts the obtained first obtained waveform and second obtained waveform. It moves the position of the time waveform such that the position of the first reflected pulse on the time axis contained in each time waveform is equal to the reference time position T_{ref}. Here, the reference time position T_{ref} is a position on the time axis previously set by a user or an automated algorithm.

The time waveform is moved such that the position on the time axis corresponding to the first reflected pulse contained in the time waveform (t₁ in FIG. 4B) is equal to the reference time position T_{ref}. After the position of the first reflected pulse in the first obtained waveform is defined as the reference time position T_{ref}, the waveform adjusting unit 107 may adjust the position of the second obtained waveform on the time axis such that the correlation between the first reflected pulse contained in the first obtained waveform and the first reflected pulse contained in the second obtained waveform is maximized.

After that, the first obtained waveform in which the position on the time axis is adjusted to the reference time position is output as a first adjusted waveform. Similarly, the second obtained waveform in which the position on the time axis is adjusted to the reference time position is output as a second adjusted waveform.

### Extraction of Time Waveform of Reflected Pulse

The waveform forming unit 108 obtains an extracted waveform by summing the first adjusted waveform and the second adjusted waveform. When the first reflection portion and the second reflection portion of the object are positioned in the mixed region A+B, the time interval Δt between the first and second reflected pulses contained in each of the first obtained waveform and the second obtained waveform changes. When the position of the first reflected pulse in each time waveform on the time axis is adjusted to the reference time position T_{ref} in the waveform adjusting unit 107, the position of the second reflected pulse in the first adjusted waveform on the time axis and the position of the second reflected pulse in the second adjusted waveform on the time axis are different.

Accordingly, a measured waveform obtained by summing the first adjusted waveform and the second adjusted waveform is a time waveform in which a signal component of the second reflected pulse is suppressed. Repeating the above-described series of processes for obtaining and adjusting time waveforms a plurality of number of times and summing one or more time waveforms in addition to the first and second time waveforms, such as third, fourth, . . . time waveforms, enables the signal component of the second reflected pulse to be further weakened and the time waveform of the first reflected pulse to be formed as a measured waveform with high accuracy. That is, the time waveform of a reflected pulse reflected from a desired interface, here, the first reflection portion can be accurately formed.

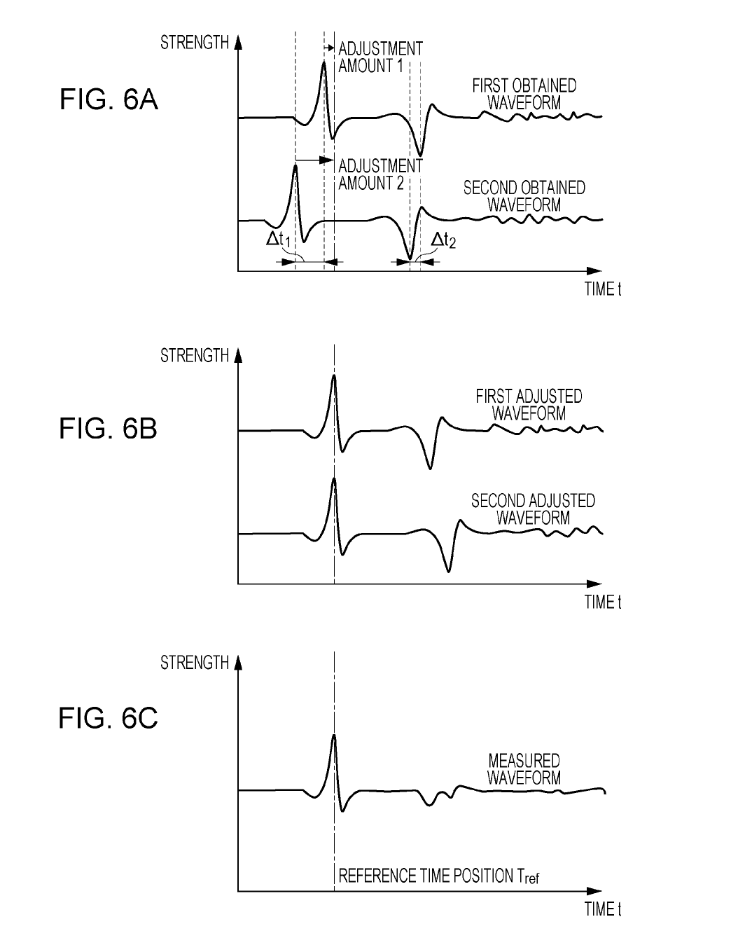

FIGS. 6A to 6C illustrate time waveforms of terahertz wave pulses from the waveform obtaining unit 105 to the waveform forming unit 108. FIG. 6A is a spectral graph of a first obtained waveform and a second obtained waveform obtained by the waveform obtaining unit 105 in the present embodiment. The first obtained waveform is a time waveform when the collection point of terahertz wave pulses is in the first collection point P₁. The second obtained waveform is a time waveform when the collection point of terahertz wave pulses is in the second collection point P₂.

FIG. 6A reveals that when the terahertz wave pulse is in the second collection point P₂, the reflection portions of the object are near the shaping unit 102 and thus the optical path length reduces. The position on the time axis of each of the first reflected pulse and the second reflected pulse contained in the second obtained waveform are shifted to the left side to that for the first obtained waveform. In other words, the reflection portions of the object are relatively near, and thus the optical path length of the terahertz wave pulse from each reflection portion reduces.

When the first reflection portion is in the collection-in-progress region and the second reflection portion is in the parallel region, that is, the object is in the mixed region, the difference Δt₁ between times of the first reflected pulses in the first obtained waveform and the second obtained waveform and the difference Δt₂ between times of the second reflected pulses therein are different. When the first reflection portion is in the collection-in-progress region A, Δt₁ is larger than Δt₂ by the amount of change in the optical path length resulting from a change in the beam shape of a reflected terahertz wave pulse reaching the generating and detecting unit 101.

FIG. 6B is an illustration for describing an adjusted waveform in the waveform adjusting unit 107. In this case, the position of each of the first and second obtained waveforms is shifted to the right side. At this time, the waveform position on the time axis is adjusted such that the position of the first reflected pulse is equal to the reference time position T_{ref}, and the first adjusted waveform illustrated in FIG. 6B is obtained. Similarly, the waveform position on the time axis of the second obtained waveform is adjusted by the waveform adjusting unit, and the second adjusted waveform illustrated in FIG. 6B is obtained. In this way, when the position on the time axis of the first reflected pulse of each time waveform is at the reference time position T_{ref}, the position on the time axis of the second reflected pulse in the first adjusted waveform and that in the second adjusted waveform are different.

FIG. 6C is an illustration for describing a measured waveform extracted in the waveform forming unit 108.

The waveform forming unit 108 extracts a time waveform in which the first adjusted waveform and the second adjusted waveform are summed. In the extracted measured waveform, signals regarding the first reflected pulse strengthen each other, whereas signals regarding the second reflected pulses weaken each other. That is, a signal relating to the first reflected pulse can be formed by changing the strength ratio between the first reflected pulse and the second reflected pulse. That is, even when there are reflected pulses of terahertz waves reflected from a plurality of interfaces, a reflected pulse of a terahertz wave from the reflection portion of interest can be extracted while a sufficient time length is maintained.

### Measuring Method for Use in Physical Property Measuring Device in Present Embodiment

A method of measuring an object for use in a physical property measuring device is described below. FIG. 3 is a flowchart of a measuring operation in the physical property measuring device in the present embodiment.

When measurement of a physical property of the object starts, a collection point of terahertz wave pulses is first adjusted to a position where the first reflected pulse is obtainable from the first and second reflection portions of the object using the shaping unit 102 (S1). Here, the position where the first reflected pulse is obtainable indicates any position where at least one of the first and second reflection portions of the object is in the parallel region B in the terahertz wave pulse (the mixed region A+B). That is, electromagnetic wave pulses are collected at the first collection point P₁ illustrated in FIG. 1.

After that, the time waveform of the reflected pulse reflected from the object using the waveform obtaining unit 105 by time-domain spectroscopy (S2). That is, the optical path length in the optical delaying unit is changed, and the time waveform (first obtained waveform) of the reflected terahertz wave pulse is obtained from a signal relating to the terahertz wave pulse detected by the detection unit.

When the first obtained waveform is obtained, the physical property measuring device 1 moves the time position of the first obtained waveform using the waveform adjusting unit 107 such that the position of the first reflected pulse in the first obtained waveform on the time axis is equal to the reference time position T_{ref}, which is the reference position, and the adjusted waveform (first adjusted waveform) is obtained (S3). Then, the obtained adjusted waveform is stored (S4).

After that, the collection point of terahertz wave pulses is moved, and it is determined whether a further time waveform is to be obtained (S5). In the present embodiment, when the first and second adjusted waveforms are stored in advance, the collection point is not moved (NO in S5), the stored first and second adjusted waveforms are summed, and the measured waveform is extracted (S7).

In contrast, when the second adjusted waveform is not stored and the collection point of terahertz wave pulses is to be changed, the collection point is adjusted by the collection point adjusting unit 106 (S6). Here, in the present embodiment, it is moved to the second collection point P₂. Here, the second collection point P₂ is the one where it is moved by a predetermined distance from the first collection point P₁. For the physical property measuring device 1 in the present embodiment, as illustrated in FIG. 5B, the collection point Z in the mixed region A+B is in the range from −0.5 mm to 0.5 mm, that is, a region that extends in 0.5 mm before and after the focal point in the collected electromagnetic wave pulses, and the collection point is moved in that range.

A region where the collection point is movable may be defined in advance, and the collection point may be randomly set in that region. Determining whether the collection point is to be moved or not may be based on determination whether measurements have been made a preset number of times that is two or more times, for example, 1000 times.

After that, a second obtained waveform having a waveform different from that of the first obtained waveform is obtained at the second collection point P₂ in a way similar to that for the first obtained waveform, and the second adjusted waveform is obtained.

Lastly, when a predetermined number of measurement times is reached, the stored first and second adjusted waveforms are summed, the measured waveform is formed (extracted) (S7), and the process is completed.

Here, the frequency resolution depends on the time length of the time waveform obtained by time-domain spectroscopy. In addition, the waveform of a time waveform positioned after the position of a peak signal of a reflected pulse in terms of time is important information from the viewpoint of increasing the accuracy of the frequency resolution. In the present embodiment, the process for the time waveform described above enables the time waveform of the reflected pulse from the reflection portion of interest to be formed without a decrease in the frequency resolution, even when there are reflected pulses of terahertz waves reflected from a plurality of interfaces. In particular, even when the gap between the interfaces is narrow and first and second reflected pulse signals being superimposed are obtained, the time waveform of reflected pulses from the reflection portion of interest can be formed in the present embodiment.

In the present embodiment, a terahertz wave pulse is used as an electromagnetic wave pulse. The use of transmission of a terahertz wave pulse facilitates identifying a physical property of the internal structure of an object at a depth of approximately 100 μm to 100 mm. The physical property of the object is obtainable by Fourier-transforming an extracted waveform and making use of the spectrum shape or a change from reference information.

In the present embodiment, the first reflection portion is disposed between the second reflection portion and the generating and detecting unit 101. Alternatively, the second reflection portion may be disposed between the first reflection portion and the generating and detecting unit 101. The object may further include a reflection portion other than the first and second reflection portions.

In the present embodiment, a terahertz wave used as an electromagnetic wave pulse is described. Other electromagnetic wave pulses, including an electromagnetic wave pulse in a frequency band of a microwave and in the far-infrared region, may also be used.

The gap between the first reflection portion and the second reflection portion can be a value that exceeds a magnitude at which a terahertz wave pulse is recognizable as a structure, an effective magnitude of approximately from 1/20λ to 1/100λ of a used wavelength λ. The used wavelength λ indicates an effective maximal wavelength in a frequency spectrum occupied by a terahertz wave pulse. In particular, in the present embodiment, the optimal maximum wavelength indicates a wavelength that is half the maximum power of the frequency power spectrum.

### Second Embodiment

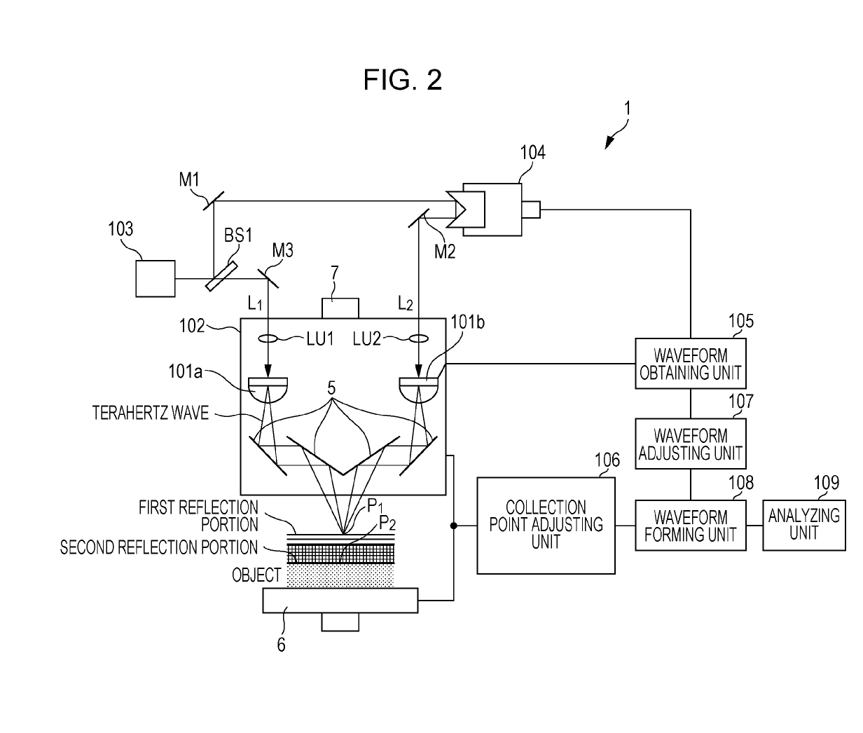

A second embodiment is distinctive in that a producing element and a detecting element for a terahertz wave are discrete and different from the first embodiment in the configuration of the portion generating and detecting a terahertz wave pulse. The second embodiment is described below with reference to FIG. 2. The description of the components common to the first embodiment is omitted.

FIG. 2 illustrates a physical property measuring apparatus according to the present embodiment.

In the present embodiment, the generating and detecting unit 101 includes two elements a generating element 101a and a detecting element 101b for respectively generating and detecting a terahertz wave. A plurality of mirrors is used as the collecting unit 5. Configuring the generating and detecting unit 101 with the generating element 101a and the detecting element 101b as separate devices can advantageously enhance the selection of a terahertz generating and detecting element. That is, different suitable elements appropriately selected based on application needs. For example, an element that has a high efficiency of outputting a terahertz wave pulse as the generating element 101a, and an element that has a high detection sensitivity as the detecting element 101b may be independently selected. Specifically, in FIG. 2, excitation light from light source 103 is split by a beam splitter BS1 into a first optical path L1 and a second optical path L2, in a manner similar to the embodiment of FIG. 1. In the present embodiment, however, excitation light passing along the optical path L₁ is directed toward the generating element 101a by the beam splitter BS1, a mirror M3, and a lens unit LU1. Excitation light passing along the optical path L₂ is directed toward the detecting element 101b by the beam splitter BS1, a series of mirrors M1 and M2 through an optical delaying unit 104 (delay unit), and a lens unit LU2. In this manner, not only the generating element 101a and the detecting element 101b can be provided as separate discrete units, but also the excitation light to generate and detect the terahertz wave pulse can be generated from separate light source units.

The shaping unit 102 in the present embodiment collects (focuses) terahertz wave pulses onto the object using four mirrors. Because the detecting element 101b and the generating element 101a are implemented as discrete (separated) units, the angle of incidence of a terahertz wave pulse incident on the object can be made variable. When the angle of incidence of a terahertz wave pulse is adjustable, a measurement region is selectable, specifically, information on the surface of the object can be a measurement target by a reduction in the angle of incidence on the object, and in contrast, information on a deep region of the object can be a measurement target by an increase in the angle of incidence. Depending on the angle of incidence of a terahertz wave pulse, the terahertz wave pulse from a specific reflection portion can be selectively avoided.

### Third Embodiment

A third embodiment is distinctive in that the physical property measuring device according to the first embodiment is applied to a tomographic apparatus and a stage that fixes an object is movable in parallel with the optical axis direction of a terahertz wave pulse. The third embodiment is described below with reference to the drawings. The description of the components common to the first embodiment is omitted.

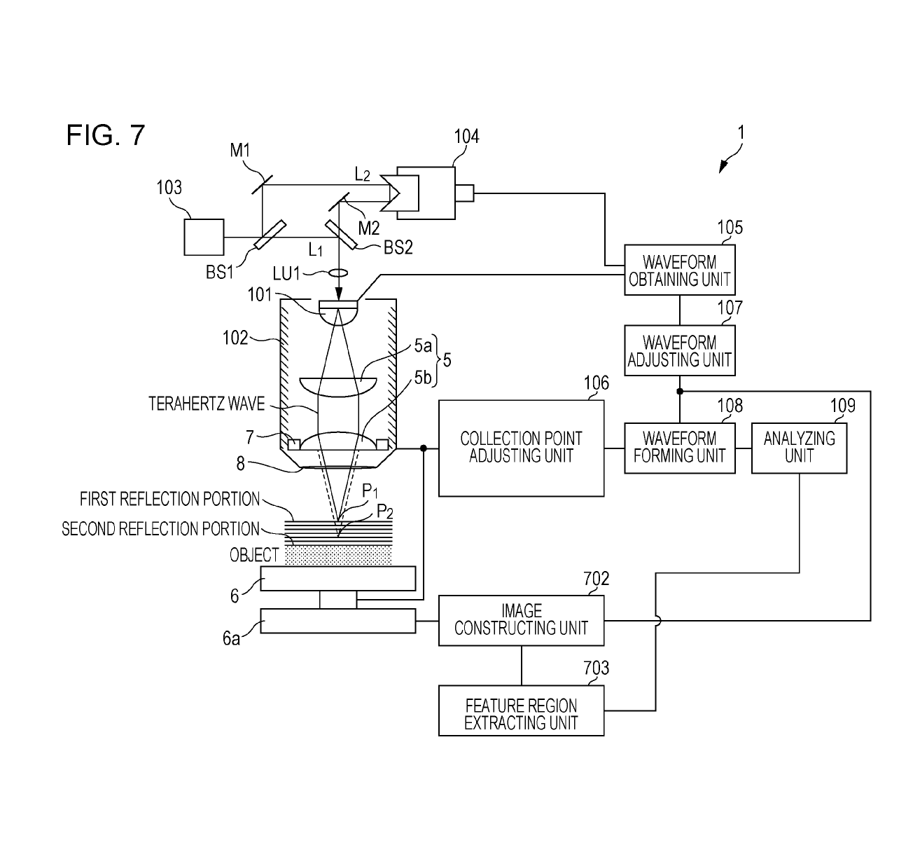

FIG. 7 illustrates a tomographic apparatus according to the present embodiment.

The time axis of the time waveform of a terahertz wave pulse can be converted into a distance. Thus the time waveform of a terahertz wave pulse can be considered to be an A scan image in a tomographic image. A B scan image and three-dimensional tomographic image are obtainable by scanning the optical axis in which a terahertz wave pulse propagates along a direction perpendicular to the direction in which the terahertz wave pulse enters the object and performing measurement.

A tomographic apparatus 1 in the present embodiment includes a movable stage 6 that is an object holding unit that relatively moves the position of each of an object and a terahertz wave pulse entering the object. The tomographic apparatus 1 further includes an image constructing unit 702 configured to construct a tomographic image of the object by matching the position of the movable stage 6 and the time waveform output from the waveform obtaining unit. The tomographic apparatus 1 further includes a feature region extracting unit 703 configured to form a feature region from the obtained tomographic image and obtains a physical property of the region extracted by the feature region extracting unit 703.

The movable stage 6 holds the object and can move it in parallel with the optical axis direction (emitting direction) of a terahertz wave pulse. The image constructing unit 702 constructs a tomographic image on the basis of the position of the movable stage 6 and a signal of a measured waveform of the waveform obtaining unit 105. A C-scan tomographic image can be constructed by two-dimensional scanning using the movable stage 6 while the optical path length difference in the optical delaying unit 104 is fixed. The image constructing unit 702 can also reconstruct a B-scan or C-scan tomographic image from the constructed three-dimensional tomographic image and output it.

The feature region extracting unit 703 forms a feature region from the tomographic image constructed by the image constructing unit 702. The feature region extracting unit 703 refers to the tomographic image for the object and selects a region of interest. The apparatus may refer to a B-scan or C-scan tomographic image and may automatically detect a position where an interface of the reflection portion is discontinuous. The boundary of a tomographic image may be established or determined from a detected discontinuous point, an image obtained by referring to the information on the boundary may be separated into several structural elements, and they may be presented.

A series of processes in a measuring operation in the tomographic apparatus 1 according to the present embodiment is described with reference to the drawings. In the present embodiment, a skin used as the object is described. The object is not limited to the skin, and various substances can be measured.

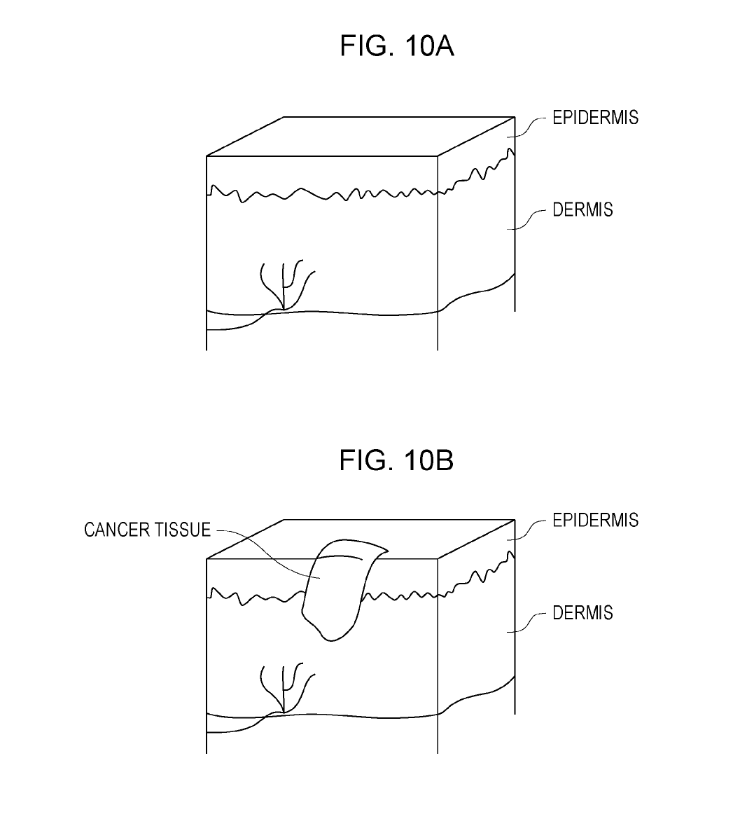

FIG. 10A is a schematic diagram of a skin used as the object in the present embodiment. A typical skin structure is the one that includes an epidermis having a thickness of several hundred micrometers and a dermis having a thickness of several millimeters. The epidermis mainly includes an epidermal cell, a pigment cell, and a Langerhans cell and has a keratin with a thickness of several tens of micrometers in the top surface. The dermis is mainly composed of collagen and elastin.

The tomographic apparatus 1 according to the present embodiment forms an image whose main targets are the epidermis, dermis, and boundary between the dermis and subcutaneous tissue. FIG. 10B is a schematic diagram when a cancer tissue exists in a skin. It has been known that the cancer tissue has a moisture content higher than that of a healthy tissue. Thus visualizing a difference between moisture contents enables identification of a cancer tissue. When a living body, typified by a skin, is used as the object, because the visible light and the infrared light has large absorption and dispersion with respect to the living body, it is difficult to obtain a tomographic image in a region of several millimeters to several tens of millimeters in the depth direction with an accuracy of several tens of micrometers to several hundred micrometers. Such a tomographic image can be obtained using an apparatus form that makes use of transmission of a terahertz wave, makes the terahertz wave have a pulsed form, and improves measurement resolution.

FIG. 9 is a flowchart that illustrates a control process in a measuring operation in the tomographic apparatus according to the present embodiment.

When the measuring operation starts, a tomographic image is obtained (S201). An observation point for a terahertz wave pulse is moved by movement of the movable stage 6, and in each observation point, the time waveform of the terahertz wave pulse is obtained by the waveform obtaining unit 105.

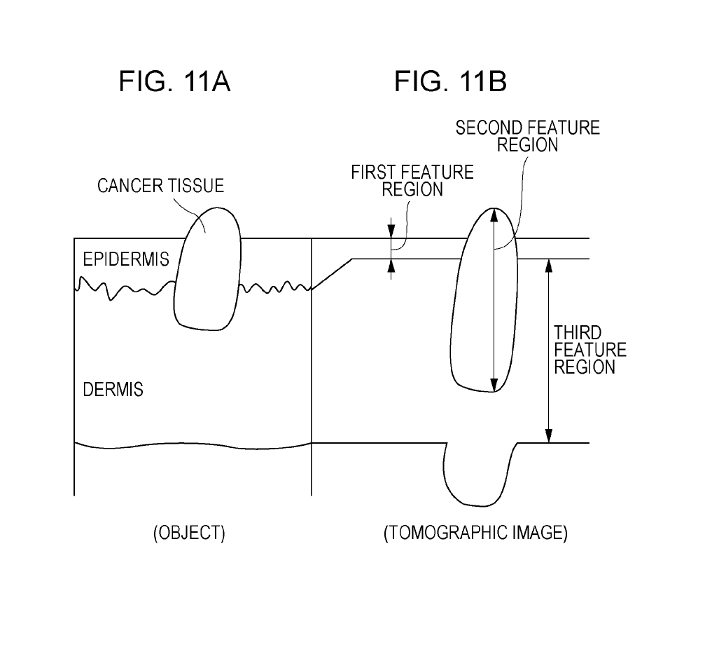

FIGS. 11A and 11B illustrate an object obtained by the tomographic apparatus according to the present embodiment. FIG. 11A is a schematic diagram of a skin that contains a cancer tissue. FIG. 11B illustrates a tomographic image after measurement of the skin containing the cancer tissue. The image constructing unit 702 constructs a tomographic image using the position of the observation point determined by the movable stage 6 and the time waveform of the terahertz wave pulse at the observation. Because the propagation speed of the terahertz wave pulse varies depending on the difference of physical properties of the sites forming the object, the optical length of each site varies. As a result, as in the tomographic image illustrated in FIG. 10A, the position of the interface partly changes, in comparison with the cross-sectional structure of the object.

The feature region extracting unit 703 selects a feature region for the constructed tomographic image (S202). For example, in the example illustrated in FIG. 11B, the region between the outermost surface of the epidermis and the interface between the epidermis and the dermis is defined as a first feature region, the region between the outermost surface of the cancer tissue and the interface between the cancer tissue and the dermis is defined as a second feature region, and the region between the interface between the epidermis and the dermis and the interface between the dermis and the subcutaneous tissue is defined as a third feature region.

The tomographic apparatus moves the observation region for a terahertz wave pulse to the feature region of interest using the movable stage 6 and an actuator 6a illustrated in FIG. 7 (S203).

The terahertz wave pulse from the interface (reflection portion) forming the feature region of interest is extracted using the steps S1 through S7 (FIG. 3) of the measuring operation used in the first embodiment, and the physical property is analyzed by analysis of the time waveform (S204). For example, when the second feature region is a target of the observation region, the time waveform from the outermost surface of the cancer tissue is extracted, and analysis of the physical property containing information on the air and the cancer tissue is conducted. After that, the time waveform from the interface between the cancer tissue and the dermis is extracted, and analysis of the physical property including information on the cancer tissue and the dermis is conducted. After that, the physical property of the cancer tissue is extracted using both of the analysis results. Here, the results of analysis of the two interfaces are used. The number of used interfaces may be one or more. If a single interface is used, the physical property of the interface itself is analyzed. This can be applied in monitoring whether the physical property of the interface of interest has changed, for example.

According to the tomographic apparatus in the present embodiment, the physical property of a feature region of interest in an obtained tomographic image of the object is measured using a measured waveform formed by the waveform forming unit. Thus the advantageous effect of being able to analyze a feature region of interest in the state where the effects of the internal structure of the object are suppressed is obtainable.

According to the tomographic apparatus in the present embodiment, the physical property of a feature region of interest in an obtained tomographic image of the object is measured using an extracted time waveform from the reflection portion. Thus the advantageous effect of being able to analyze a feature region of interest in the state where the effects of the internal structure of the object are suppressed is obtainable.

### Fourth Embodiment

A fourth embodiment is distinctive in that a correction unit configured to correct a tomographic image using obtained physical property information, and other configurations are substantially the same as in the third embodiment. The fourth embodiment is described below with reference to the drawings. The description of the configurations common to the third embodiment is omitted.

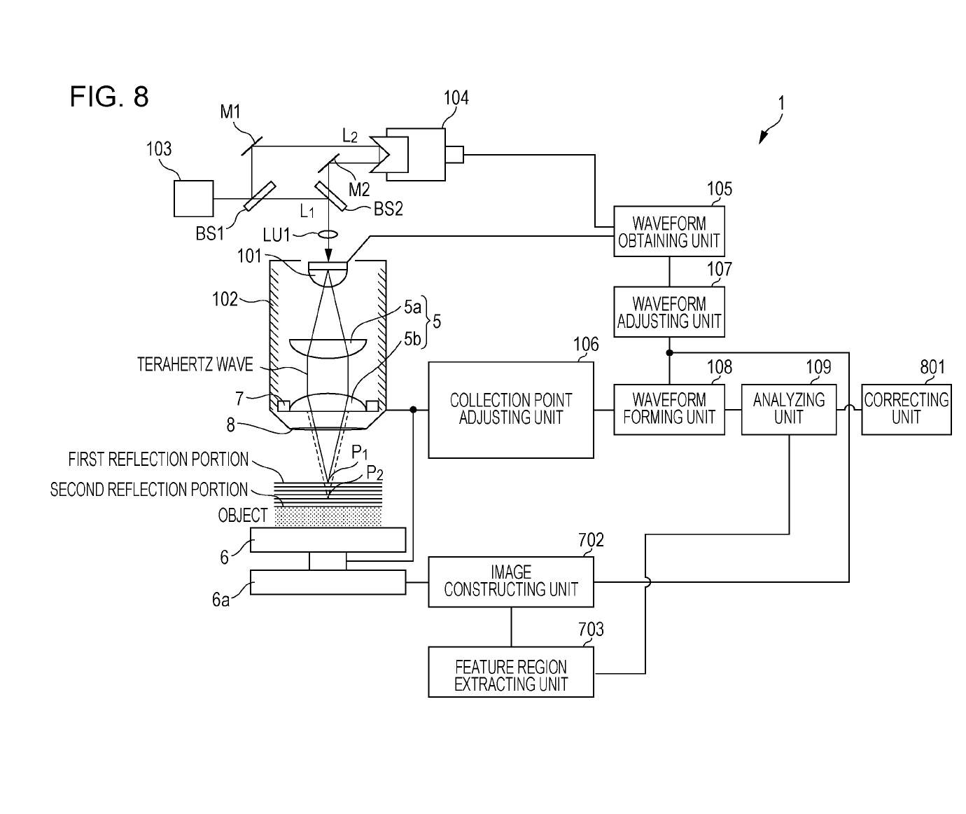

FIG. 8 illustrates a tomographic apparatus according to the present embodiment. The tomographic apparatus 1 according to the present embodiment includes a correcting unit 801 configured to correct a tomographic image using obtained physical property information. The correcting unit 801 refers to physical property information obtained in the analyzing unit 109 and adjusts the thickness of each feature region.

The tomographic apparatus according to the present embodiment first analyzes a physical property of a feature region of interest of the object using the above-described measuring method. Then, the tomographic apparatus corrects the tomographic image using the correcting unit 801 employing obtained physical property information on the interface with the feature region.

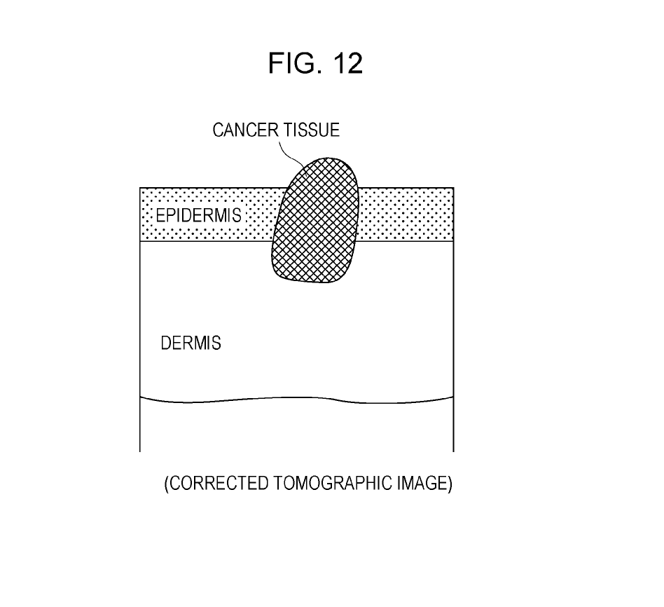

FIG. 12 illustrates a tomographic image corrected by the tomographic apparatus according to the present embodiment. The tomographic image illustrated in FIG. 12 is the one in which the tomographic image obtained in FIG. 11B is corrected. The tomographic apparatus according to the present embodiment adjusts the optical length of the obtained tomographic image. The adjustment in the optical length of the tomographic image enables an image near the object to be obtained. At this time, it is presented with the displayed form of the feature region changed depending on the physical property of each of the feature regions.

The obtained tomographic image of the object is corrected using the physical property information on the feature region obtained using the extracted time waveform from the reflection portion. Visualizing the amount of the correction facilitates obtaining the distribution of physical property information inside the object.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-034396 filed Feb. 20, 2012, which is hereby incorporated by reference herein in its entirety.

What is claimed is:

1. A measuring device for measuring a physical property of an object which is irradiated with an electromagnetic wave pulse, the object including a first reflection portion and a second reflection portion, the measuring device comprising:
a detecting unit configured to detect the electromagnetic wave pulse;
an optical delaying unit configured to delay an optical path length of excitation light reaching the detecting unit or the electromagnetic wave pulse;
a collecting unit configured to collect the electromagnetic wave pulses to a collection point;
a position adjusting unit configured to adjust a positional relationship between the object and the collection point such that a depth of focus of the electromagnetic wave pulse is in at least one of the first reflection portion and the second reflection portion of the object;
a waveform obtaining unit configured to change the optical path length in the optical delaying unit, obtain a time waveform from a signal relating to the electromagnetic wave detected by the detecting unit, obtain a first obtained waveform at a first collection point where the depth of focus of the electromagnetic wave pulse is adjusted by the position adjusting unit so as to be in only one of the first reflection portion and the second reflection portion, and obtain a second obtained waveform at a second collection point different from the first collection point; and
a waveform forming unit configured to form a measured waveform based on the first obtained waveform and the second obtained waveform.

2. The measuring device according to claim 1, wherein the depth of focus is a region that extends at least 0.5 millimeters before and after a focal point in the collected electromagnetic wave pulses.

3. The measuring device according to claim 1, wherein the waveform forming unit forms the measured waveform by superimposing first and second adjusted waveforms in which positions of first and second reflected pulses on a time axis are adjusted to respective reference positions, the first and second reflected pulses being the electromagnetic waves reflected from the first and second reflection portions in the first and second obtained waveforms.

4. The measuring device according to claim 1, wherein the waveform obtaining unit obtains one or more time waveforms in addition to the first obtained waveform and the second obtained waveform, and
the waveform forming unit forms the measured waveform based on the first obtained waveform, the second obtained waveform, and the one or more time waveforms.

5. The measuring device according to claim 1, wherein the second collection point is one at which the depth of focus of the electromagnetic wave pulse is in one of the first reflection portion and the second reflection portion in the object.

6. The measuring device according to claim 1, wherein the position adjusting unit moves the collection point where the electromagnetic wave pulses are collected with respect to the object being fixed.

7. The measuring device according to claim 1, wherein the position adjusting unit is a stage that holds the object, the object being movable in a direction parallel to an optical axis of the electromagnetic wave pulse.

8. The measuring device according to claim 1, wherein the electromagnetic wave pulse contains a part of a frequency band of 30 GHz to 30 THz.

9. A measuring method for use in a measuring device for measuring a physical property of an object, the object including a first reflection portion and a second reflection portion, the measuring device including:
a detecting unit configured to detect an electromagnetic wave pulse;
an optical delaying unit configured to delay an optical path length of excitation light reaching the detecting unit;
a collecting unit configured to collect the electromagnetic wave pulses to a collection point;
a position adjusting unit configured to adjust a positional relationship between the object and the collection point such that a depth of focus of the electromagnetic wave pulse is in at least one of the first reflection portion and the second reflection portion of the object;
a waveform obtaining unit configured to change the optical path length in the optical delaying unit and obtain a time waveform from a signal relating to the electromagnetic wave detected by the detecting unit,
the measuring method comprising:
obtaining a first obtained waveform at a first collection point where the depth of focus of the electromagnetic wave pulse is adjusted by the position adjusting unit so as to be in only one of the first reflection portion and the second reflection portion and obtaining a second obtained waveform at a second collection point different from the first collection point; and
adjusting positions of first reflected pulses reflected from the first reflection portion in the first and second obtained waveforms on a time axis to respective reference positions to form first and second adjusted waveforms and forming a measured waveform by summing the first and second adjusted waveforms.

10. The measuring method according to claim 9, further comprising Fourier-transforming the extracted measured waveform and obtaining a spectrum of the object.

11. A tomographic apparatus for obtaining a tomographic image of an object, the object including a first reflection portion and a second reflection portion, the tomographic apparatus comprising:
a detecting unit configured to detect an electromagnetic wave pulse;
an optical delaying unit configured to delay excitation light reaching the detecting unit;
a collecting unit configured to collect the electromagnetic wave pulses to a collection point;
a position adjusting unit configured to perform movement in parallel with an optical axis of the electromagnetic wave pulse in the collection point with respect to the object such that a depth of focus of the electromagnetic wave pulse is in at least one of the first reflection portion and the second reflection portion of the object;
a waveform obtaining unit configured to make the optical path length in the optical delaying unit variable, obtain a time waveform from information on the electromagnetic wave detected by the detecting unit, obtain a first obtained waveform at a first collection point where the depth of focus of the electromagnetic wave pulse is adjusted by the position adjusting unit so as to be in only one of the first reflection portion and the second reflection portion, and obtain a second obtained waveform at a second collection point different from the first collection point;
a waveform forming unit configured to form a measured waveform based on the first obtained waveform and the second obtained waveform;
a stage that holds the object and that relatively moves the object and the position of the electromagnetic wave pulse; and
an image constructing unit configured to construct a tomographic image of the object based on the position of the stage and the measured waveform formed by the waveform forming unit.

12. The tomographic apparatus according to claim 11, wherein the waveform forming unit forms the measured waveform by superimposing first and second adjusted waveforms in which positions of first and second reflected pulses on a time axis are adjusted to respective reference positions, the first and second reflected pulses being the electromagnetic waves reflected from the first and second reflection portions in the first and second obtained waveforms.

13. A measuring device for measuring a physical property of an object which is irradiated with an electromagnetic wave pulse, the object including a first reflection portion and a second reflection portion, the measuring device comprising:
a detecting unit configured to detect the electromagnetic wave pulse;
an optical delaying unit configured to delay an optical path length of excitation light reaching the detecting unit or the electromagnetic wave pulse;
a collecting unit configured to collect the electromagnetic wave pulses to a collection point;
a position adjusting unit configured to adjust a positional relationship between the object and the collection point such that a depth of focus of the electromagnetic wave pulse is in at least one of the first reflection portion and the second reflection portion of the object;
a waveform obtaining unit configured to change the optical path length in the optical delaying unit, obtain a time waveform from a signal relating to the electromagnetic wave detected by the detecting unit, obtain a first obtained waveform at a first collection point, and obtain a second obtained waveform different from the first obtained waveform at a second collection point different from the first collection point; and
a waveform forming unit configured to form a measured waveform based on the first obtained waveform and the second obtained waveform.

14. The measuring device according to claim 13, wherein the waveform forming unit forms the measured waveform by superimposing first and second adjusted waveforms in which positions of first and second reflected pulses on a time axis are adjusted to respective reference positions, the first and second reflected pulses being the electromagnetic waves reflected from the first and second reflection portions in the first and second obtained waveforms.
